(19) United States
(12) Patent Application Publication
(10) Pub. No.: US 2025/0022608 A1
(43) Pub. Date: Jan. 16, 2025

(54) INTELLIGENCE BASED ON MORPHOLOGICAL AND HEMODYNAMIC FACTORS OF ANEURYSM

(71) Applicants: INDUSTRY-UNIVERSITY COOPERATION FOUNDATION HANYANG UNIVERSITY ERICA CAMPUS, Ansan-si (KR); UIF (University Industry Foundation), Yonsei University, Seoul (KR); CATHOLIC KWANDONG UNIVERSITY INDUSTRY COOPERATION FOUNDATION, Gangneung-si (KR)

(72) Inventors: Je Hoon OH, Anyang-si (KR); Hyeondong YANG, Geoje-si (KR); Yong Bae KIM, Seoul (KR); Jung-Jae KIM, Seoul (KR); Kwang-Chun CHO, Seoul (KR)

(21) Appl. No.: 18/900,955

(22) Filed: Sep. 30, 2024

Related U.S. Application Data
(63) Continuation of application No. PCT/KR2023/004488, filed on Apr. 4, 2023.

(30) Foreign Application Priority Data
Apr. 5, 2022 (KR) 10-2022-0041961

(51) Int. Cl. G16H 50/30; A61B 5/02; G16H 30/40; G16H 50/70

(52) U.S. Cl. CPC G16H 50/30; A61B 5/02014; G16H 30/40; G16H 50/70

(57) ABSTRACT

The present invention relates to a method and device for predicting aneurysm rupture using artificial intelligence based on morphological and hemodynamic factors of aneurysms. The method for predicting aneurysm rupture according to one embodiment of the present disclosure may comprises acquiring an image of a blood vessel; deriving moment of inertia based on the image of the blood vessel; acquiring a hemodynamic factor; outputting the rupture risk when the moment of inertia and the hemodynamic factor are inputted to a pre-trained artificial neural network; and predicting the possibility of rupture as possible when the rupture risk is greater than a predetermined rupture threshold value, and predicting the possibility of rupture as absent when the rupture risk is not greater than the predetermined rupture threshold value.

### CROSS-REFERENCE TO RELATED APPLICATIONS

This application is a PCT Continuation By-Pass application of PCT Application No. PCT/KR2023/004488 filed on Apr. 4, 2023, in the Korean Intellectual Property Office, the entire disclosures of which are incorporated herein by reference for all purposes.

### BACKGROUND

### 1. Field

The present disclosure relates to a method for predicting an aneurysm rupture and a device thereof. More particularly, the present disclosure relates to a method and device for predicting aneurysm rupture using artificial intelligence based on morphological factors and hemodynamic factors of an aneurysm.

### 2. Description of the Related Art

An aneurysm is a pocket formed in an artery, which means that a part of the blood vessel stretches and looks like a balloon. An aneurysm can occur anywhere in the arteries in our body, such as the brain, heart, and lower limbs.

In particular, cerebral aneurysm refers to a disease in which blood vessels in the brain swell or expand like a balloon, and is classified into small, large, and super types according to their size. A cerebral aneurysm rupture can cause serious health problems such as subarachnoid hemorrhage, symptoms such as nausea and vomiting, loss of consciousness, coma and even death.

However, since most of the non-ruptured cerebral aneurysm do not cause symptoms, it is important to perform an appropriate image test to diagnose the occurrence of cerebral aneurysm, and further evaluate the possibility of cerebral aneurysm rupture to prevent unnecessary treatment to determine whether to treat them.

Methods of diagnosing cerebral aneurysm include ultrasound examination, computed tomography (CT), computed tomography angiography (CT angiography), magnetic resonance imaging (MRI), magnetic resonance angiography (MR angiography), angiography, etc.

A conventional method of diagnosing the possibility of cerebral aneurysm rupture is to use a volume among morphological factors calculated through image data of the cerebral aneurysm. In general, the greater the volume of the cerebral aneurysm, the greater the size, so the possibility of rupture may be high.

However, since many cerebral aneurysms have very complex shapes, it is often impossible to determine the shape of a cerebral aneurysm simply by its volume.

Another conventional method of diagnosing a cerebral aneurysm is to determine the aspect ratio of the cerebral aneurysm. In general, a large horizontal and vertical ratio indicates that the size has enlarged on one side, so it may be highly likely to rupture. However, there are limitations to determining the possibility of rupture with only the horizontal and vertical ratios for cerebral aneurysm of complex shapes.

Accordingly, there is an increasing demand for a method for more accurately predicting the probability of cerebral aneurysm rupture by using a morphological factor of the cerebral aneurysm.

### SUMMARY

The technical problem that the present disclosure intends to solve is to provide a method and a device for deriving a moment of inertia using morphological factors of an aneurysm, obtaining the aneurysm rupture risk by inputting the moment of inertia together with a hemodynamic factor to an artificial neural network, and predicting a possibility of aneurysm rupture based on the rupture risk.

The technical problems of the present disclosure are not limited to the technical problems mentioned above, and other technical problems that are not mentioned will be clearly understood by those skilled in the art from the following description.

A method for predicting aneurysm rupture according to one embodiment of the present disclosure for achieving the above-described technical objectives may comprises acquiring an image of a blood vessel; deriving moment of inertia based on the image of the blood vessel; acquiring a hemodynamic factor; outputting the rupture risk when the moment of inertia and the hemodynamic factor are inputted to a pre-trained artificial neural network; and predicting the possibility of rupture as possible when the rupture risk is greater than a predetermined rupture threshold value, and predicting the possibility of rupture as absent when the rupture risk is not greater than the predetermined rupture threshold value.

According to the present disclosure as described above, the rupture risk can be derived by inputting the morphological factor and the hemodynamic factor of the aneurysm to the artificial neural network, and the possibility of rupture can be predicted based on the derived risk.

In addition, according to the present disclosure as described above, the moment of inertia of the aneurysm is derived based on the morphological factor and is used, thereby having an effect of more accurately predicting the possibility of aneurysm rupture having an atypical shape.

### BRIEF DESCRIPTION OF THE DRAWINGS

FIG. 1 is a block diagram of a device for predicting aneurysm rupture according to an embodiment of the present disclosure.

FIG. 2 is an example for explaining a method of deriving a moment of inertia of an aneurysm, according to an embodiment of the present disclosure.

FIG. 3 is an example for explaining a difference in effect between the conventional method and the present disclosure according to an embodiment of the present disclosure.

FIG. 4 is a flowchart of a method of predicting aneurysm rupture according to an embodiment of the present disclosure.

FIG. 5 is a flowchart of a method of deriving a moment of inertia of an aneurysm, according to an embodiment of the present disclosure.

FIG. 6 is a configuration diagram of an artificial neural network according to an embodiment of the present disclosure.

FIG. 7 is an illustration of a receiver operating characteristic (ROC) curve, according to an embodiment of the present disclosure.

### DETAILED DESCRIPTION

The advantages and features of the present disclosure, as well as methods for achieving them, will become apparent with reference to the embodiments described below in conjunction with the accompanying drawings. However, the present disclosure is not limited to the embodiments disclosed herein, and may be implemented in various other forms. The embodiments are merely provided to ensure that the present disclosure is fully understood, and to fully convey the scope of the invention to those skilled in the art. The present invention is defined only by the scope of the claims.

A brief explanation of the terminology used herein will be provided, followed by a detailed description of the present disclosure.

The terms used in the present disclosure have been selected, whenever possible, from commonly used terms that best reflect the functions within the scope of the present invention. However, the meaning of such terms may vary depending on the intent of those skilled in the art, precedents, or the emergence of new technologies. In certain cases, terms selected arbitrarily by the applicant may be used, in which case their meanings will be described in detail in the corresponding portion of the description of the invention. Therefore, the terms used herein should not be understood merely as the names of terms, but should be interpreted based on the meanings they convey and the overall context of the present disclosure.

Throughout the specification, when a portion “includes” a certain component, it implies that other components may also be included unless specifically stated otherwise. Furthermore, the terms “processor”, “unit”, “module”, or “part” as used herein, refer to units that perform at least one function or operation, and may be implemented as hardware components such as software, FPGA, or ASIC, or as a combination of software and hardware. However, the terms “processor”, “unit”, “module”, or “part” are not limited to software or hardware alone. These terms may also refer to components stored on an addressable storage medium and configured to reproduce one or more processors. Thus, for example, the terms “processor”, “unit”, “module”, or “part” may refer to software components, object-oriented software components, class components, and task components, as well as processes, functions, attributes, procedures, subroutines, program code segments, drivers, firmware, microcode, circuits, data, databases, data structures, tables, arrays, and variables.

The following is a detailed description of embodiments of the present disclosure, provided with reference to the accompanying drawings, so that those skilled in the art may easily practice the invention. For clarity, irrelevant portions are omitted in the drawings.

The terms including ordinal numbers such as “first” and “second” may be used to describe various components, but such components are not limited by these terms. The terms are used merely to distinguish one component from another. For example, a first component may be referred to as a second component without departing from the scope of the present disclosure, and similarly, a second component may be referred to as a first component. The term “and/or” includes any and all combinations of one or more of the associated listed items.

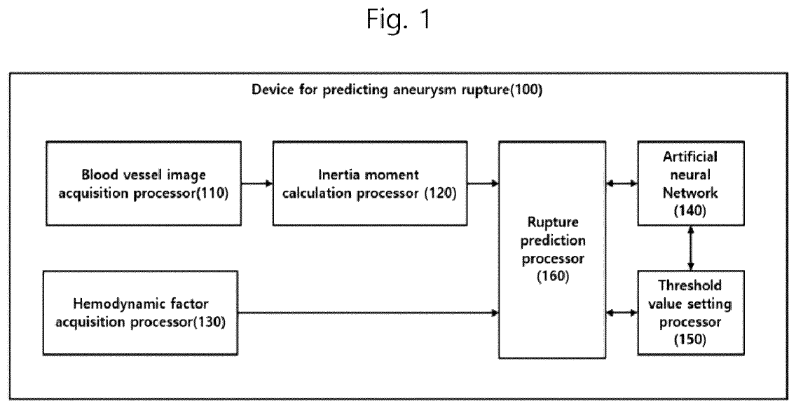

Hereinafter, an embodiment of the present disclosure will be described with reference to FIGS. 1 to 7.

FIG. 1 is a block diagram of a device for predicting aneurysm rupture according to an embodiment of the present disclosure. A configuration of a device for predicting aneurysm rupture according to an embodiment of the present disclosure will be described with reference to FIG. 1.

The device for predicting aneurysm rupture 100 according to an embodiment of the present disclosure may include a blood vessel image acquisition processor 110, an inertia moment calculation processor 120, a hemodynamic factor acquisition processor 130, a rupture prediction processor 160, an artificial neural network 140, and a threshold value setting processor 150.

The blood vessel image acquisition processor 110 may acquire an image including a blood vessel in which an aneurysm has occurred.

The aneurysm described in the present disclosure may be a cerebral aneurysm.

The image may be acquired by a diagnosis using an ultrasound examination, a computed tomography (CT), a computed tomography angiography (CT angiography), a magnetic resonance imaging (MRI), a magnetic resonance angiography (MR. angiography), or a angiography. The diagnostic method is illustrative and is not limited thereto.

The image may include a 2-dimension (2D) image or a 3-dimension (3D) image.

The inertia moment calculation processor 120 may calculate and derive the moment of inertia of the aneurysm.

The moment of inertia is a physical quantity that represents the size of an object in physics that is rotating to continue its rotation.

In physics, the scalar moment of inertia (I) for a point mass rotating about a given axis is defined as in Equation 1 below.

$$I = {mr}^{2} \qquad [ Equation 1 ]$$

Here, m is a mass, and r is a distance from the rotation axis to the point mass.

For n point masses rotating about the same axis, the total moment of inertia is the sum of the moment of inertia of each point mass as shown in Equation 2 below.

$$I = {m}_{1} {r}_{1}^{2} + {m}_{2} {r}_{2}^{2} + … + {m}_{n} {r}_{n}^{2} = {\sum }_{i = 1}^{n} {m}_{i} {r}_{i}^{2} \qquad [ Equation 2 ]$$

When an arbitrary mass other than point masses is distributed in a space at a density ρ(r), the sum of each point mass is changed into an integral, and the moment of inertia may be defined as in Equation 3 below.

$$I = \int {r}^{2} dm = {\int }_{V} {r}^{2} ρ ( r ) dV \qquad [ Equation 3 ]$$

According to an embodiment of the present disclosure, a moment of inertia is used to numerically express the morphological factor of an aneurysm. Conventionally, the morphological factor of the aneurysm was used in volume, aspect ratio, and the like, but these values were insufficient to accurately predict aneurysm rupture having various shapes.

When the morphological value of the aneurysm is expressed as the moment of inertia, it is possible to compare how far the point mass of the aneurysm is from the central axis. A large moment of inertia at similar masses means that the point mass is farther away from the central axis. An aneurysm having a larger moment of inertia can be meant to be a more swollen shape. Since aneurysm with a large degree of swelling are more likely to rupture, the moment of inertia can be used to predict the possibility of rupture.

Using the moment of inertia, the possibility of aneurysm rupture of a complex shape can be accurately predicted. There is a limit to the conventional method of predicting the possibility of aneurysm rupture of a complex shape based on the volume or aspect ratio. In contrast, the moment of inertia can accurately show how swollen the aneurysm is in a complex shape, and thus the possibility of rupture can be more accurately predicted.

In addition, rather than using the moment of inertia alone, factors related to the shape of the blood vessel and the aneurysm including a diameter of the blood vessel, a volume of the aneurysm, a length and width of the aneurysm, and a Neck (aneurysm entrance surface) area are used to normalize the moment of inertia through Equation 4 below to be used to predict the possibility of rupture.

$$Normalizing the moment of inertia = \frac{The moment of inertia}{Mass or Neck area or Diameter of blood vessel} \qquad [ Equation 4 ]$$

In addition, the moment of inertia may be calculated through Equation 5 below by using not only r2 (the square of the distance) but also r3 (Skewness) or r4 (kurtosis).

$$I ( skewness ) = \int {r}^{3} dm or I ( kurtosis ) = \int {r}^{4} dm \qquad [ Equation 5 ]$$

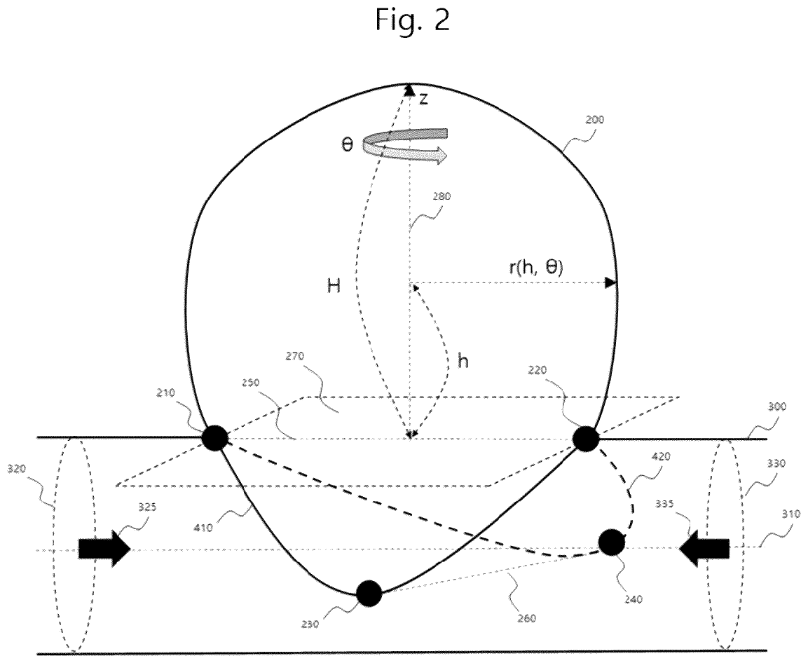

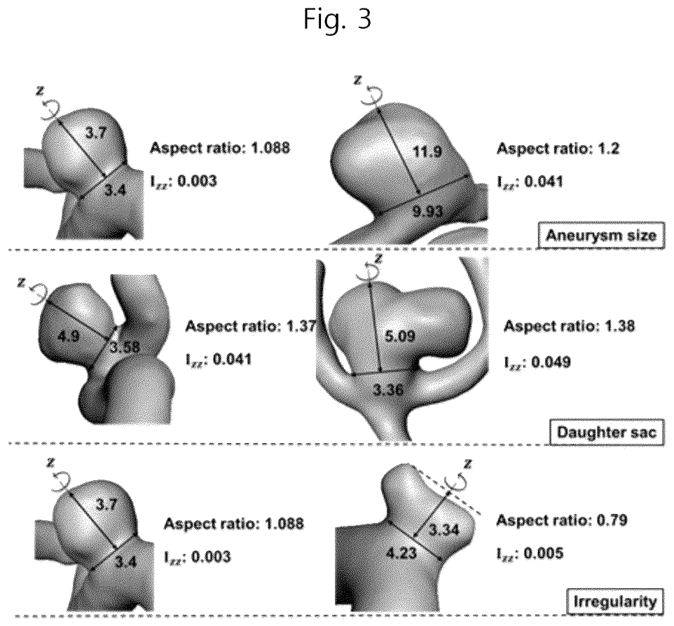

The method of deriving the moment of inertia of the aneurysm will be described in detail with reference to FIGS. 2 and 3.

The hemodynamic factor acquisition processor 130 may acquire a hemodynamic factor of a blood vessel included in the blood vessel image.

The hemodynamic factor may include at least one of blood flow rate, systolic blood pressure, diastolic blood pressure, and vascular wall elasticity, but these are just examples, and the present disclosure is not limited thereto.

The hemodynamic factor acquisition processor 130 may acquire the hemodynamic factor through hemodynamic monitoring. hemodynamic monitoring is the observation of hemodynamic variables (blood pressure, heart rate, etc.) over time. The blood pressure may be monitored in an invasive manner using a blood pressure transducer assembly or may be repeatedly measured using a blood pressure cuff that is used by being swollen with air in a non-invasive manner.

The hemodynamic factor acquisition processor 130 may receive the hemodynamic factor from the hemodynamic monitoring device in a wired or wireless manner. Alternatively, the numerical value of the hemodynamic factor may be directly input from a user who uses the device for predicting aneurysm rupture 100.

Since acquiring the hemodynamic factor is a technique that is obvious to those skilled in the art to which the present disclosure belongs, a detailed description thereof will be omitted.

The artificial neural network 140 is a neural network trained to output the rupture risk based on previously collected data of the moment of inertia, the hemodynamic factor, and the possibility of rupture.

An artificial neural network (ANN) is a machine learning algorithm created by imitating the principles and structures of a human neural network. It mimics a process in which neurons in the human brain receive a certain signal, stimulus, or the like, and transmit a result signal when the stimulus exceeds a certain threshold. The artificial neural network may train the neural network through a process of adjusting an internal weight using backpropagation or the like.

When receiving the moment of inertia and the hemodynamic factor, the artificial neural network 140 according to an embodiment of the present disclosure may output the rupture risk. The rupture risk may be a value between 0 and 1.

In order to train the artificial neural network 140 in advance, each moment of inertia and hemodynamic factor corresponding to the rupture risk of 0 or 1 may be collected and used for training.

The artificial neural network 140 may include any one of an ANN, a deep neural network (DNN), a convolution neural network (CNN), a recurrent neural network (RNN), a long short-term memory (LSTM), or gated recurrent units (GRU), and these are only examples and are not limited thereto.

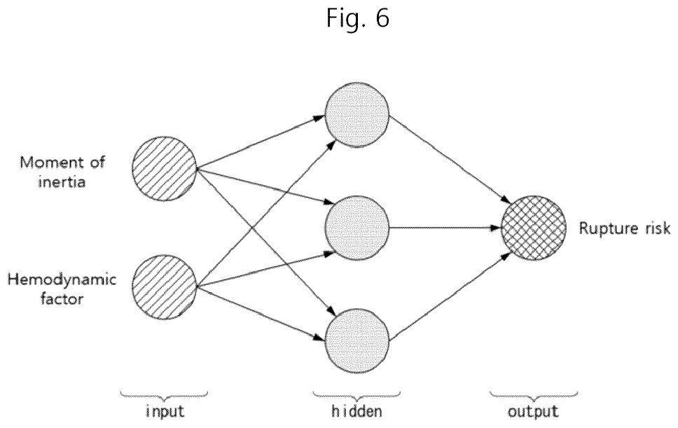

The operation of the artificial neural network 140 will be described in detail in the description of FIG. 6.

The threshold value setting processor 150 may set a rupture threshold value, which is a criterion for determining the possibility of rupture, based on the degree of rupture risk output from the artificial neural network 140.

The threshold value setting processor 150 may derive the rupture threshold value based on a receiver operating characteristic (ROC) curve generated using the trained artificial neural network 140.

The ROC curve is a graph showing the performance of the classification model at all classification thresholds, in which the x-axis is FPR (1-specificity) and the y-axis is TPR (sensitivity). The ROC curve is a graph expressing the relationship between sensitivity and specificity. The ROC curve evaluates the performance of the model using AUC (Area Under Curve: area under the graph). The larger the AUC, the more accurate it is.

The ROC curve may be used as a means for evaluating the performance of the AI model. A large AUC of the ROC curve may mean that the output of the AI model is accurate. For example, if the AI model that classifies cancer classifies cancer as cancer and non-cancer as non-cancer, the AUC is 1, but if it classifies cancer as non-cancer or non-cancer as cancer, the AUC can range between 0 and 1.

The threshold value setting processor 150 may input the previously collected moment of inertia and the hemodynamic factor to the trained artificial neural network 140 and generate the ROC curve based on the output.

The threshold value setting processor 150 may derive a point of the ROC curve closest to the coordinates (0, 1) of the ROC curve graph, and may set the sensitivity of the point as the rupture threshold value.

The threshold value setting processor 150 may obtain an intersecting point at which the ROC curve and a straight line having a slope of 1 intersect each other, and derive a largest value among sensitivities of the intersecting point as the rupture threshold value.

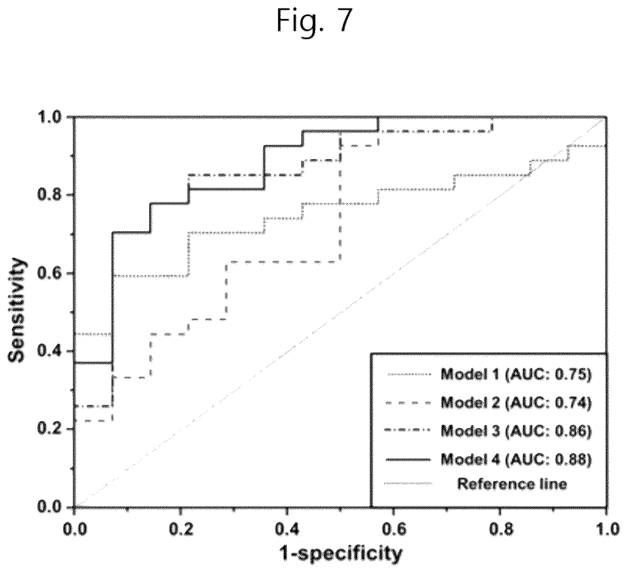

The operation of the threshold value setting processor 150 will be described in detail with reference to FIG. 7.

The rupture prediction processor 160 may input the moment of inertia derived by the inertia moment calculation processor 120 and the hemodynamic factor acquired by the hemodynamic factor acquisition processor 130 to the artificial neural network 140, and determine the possibility of aneurysm rupture by comparing the degree of rupture risk output from the artificial neural network 140 with the rupture threshold value derived by the threshold value setting processor 150.

Since the rupture risk has a value of 0 to 1, the rupture prediction processor 160 may use the rupture threshold value as a reference to determine the possibility of rupture.

When the rupture risk is greater than the rupture threshold value, the rupture prediction processor 160 may determine that there is a possibility of rupture. When the rupture risk is not greater than the rupture threshold value, the rupture prediction processor 160 may determine that there is no possibility of rupture.

FIG. 2 is an example for explaining a method of deriving a moment of inertia of an aneurysm, according to an embodiment of the present disclosure. A method of deriving a moment of inertia of an aneurysm will be described with reference to FIG. 2.

FIG. 2 is an example simply illustrating a blood vessel 300 and an aneurysm 200 included in a blood vessel image for description.

The inertia moment calculation processor 120 may derive intersecting surfaces 410 and 420 at which the blood vessel 300 and the aneurysm 200 intersect. The intersecting surfaces 410 and 420 may be curved surfaces generated along a point where the blood vessel 300 and the aneurysm 200 are in contact with each other.

The inertia moment calculation processor 120 derives the centerline 310 of the blood vessel 300. The centerline 310 may be a virtual line passing through the center of the blood vessel 300.

The inertia moment calculation processor 120 may derive the first point 210 where the first cross-section 320, which is a virtual cross-section perpendicular to the centerline 310, first meets the intersecting surface 410 and 420 when the first cross-section 320 moves in the first direction 325 along the centerline 310. The first direction 325 may be a direction from one end of the blood vessel 300 toward the aneurysm 200. As illustrated in FIG. 2, for convenience of description, it is assumed that a direction from left to right is a first direction 325.

The inertia moment calculation processor 120 may derive a second point 220 where the second cross-section 330, which is a virtual cross-section perpendicular to the centerline 310, first meets the intersecting surface 410 and 420 when the second cross-section 330 moves in the second direction 335 along the centerline 310. The second direction 335 may be a direction different from the first direction 325 and may be a direction from the other end of the blood vessel 300, which is different from the one end of the blood vessel 300 toward the aneurysm 200. The second direction 335 may be a direction opposite to the first direction 325. For convenience of description, the second direction 335 may be a direction from right to left.

The inertia moment calculation processor 120 may derive a virtual first line 250 connecting the first point 210 and the second point 220.

The inertia moment calculation processor 120 may derive a third point 230 and a fourth point 240, which are farthest from the first line 250 among points on the intersecting surfaces 410 and 420. The third point 230 may be a point located at an edge of any one surface of the intersecting surfaces 410 and 420 divided by the first line 250. For convenience of description, the third point 230 may be a point farthest from the first line 250 among points located on the first intersecting surface 410. The fourth point 240 may be a point located at an edge of a surface different from a surface where the third point 230 is located among the intersecting surfaces 410 and 420 divided by the first line 250. For convenience of description, the fourth point may be a point farthest from the first line 250 among points located on the second intersecting surface 420.

The inertia moment calculation processor 120 may derive a virtual second line 260 connecting the third point 230 and the fourth point 240.

The inertia moment calculation processor 120 may derive a virtual cross section parallel to the second line 260 and including the first line 250 as a bottom surface 270 of the aneurysm 200.

The inertia moment calculation processor 120 may derive the moment of inertia (IZ) of the aneurysm 200 around a rotation axis 280 perpendicular to the bottom surface 270.

When the distance r(h, θ) is set with respect to the point mass m located at the height h and the plane direction angle θ with respect to the vertical rotation axis 280, the inertia moment calculation processor 120 may derive the moment of inertia (Iz) by the following Equation 6.

$${I}_{z} = {\sum }_{h = 0}^{H} {\sum }_{θ = 0}^{2 π} m {\{ r ( h , θ ) \}}^{2} \qquad [ Equation 6 ]$$

According to an embodiment of the present disclosure, it is assumed that the mass of the aneurysm is almost the same clinically, and the moment of inertia (Iz) may be derived using the average mass of the aneurysm. In order to predict the aneurysm rupture, since the degree of swelling is more important than the mass of the aneurysm, the moment of inertia (Iz) may be derived by deriving a distance r(h,θ) rather than a mass from a blood vessel image.

According to another exemplary embodiment of the present disclosure, the moment of inertia (Iz) may be derived as an integral with respect to the amount of the point mass m and the distance r(h, θ).

According to another embodiment of the present disclosure, the inertia moment calculation processor 120 may assume an x-axis and a y-axis arbitrarily orthogonal on the bottom surface 270, and may derive the moment of inertia Ix of the aneurysm 200 about the x-axis as a rotation axis or may derive the moment of inertia Iy of the aneurysm 200 about the y-axis as a rotation axis.

FIG. 3 is an example for explaining a difference in effect between the conventional method and the present disclosure according to an embodiment of the present disclosure.

Conventional methods of predicting aneurysm rupture have used the aspect ratio of the aneurysm.

Referring to the first line of FIG. 3, when the aspect ratio is larger, the moment of inertia may also be larger, and in this case, the prediction of the aneurysm may be the same as that of the conventional method.

Referring to the second line of FIG. 3, it can be seen that there is little difference in the aspect ratio, but the difference in the moment of inertia is larger, and it may be more accurate to predict aneurysm rupture based on the moment of inertia since the right aneurysm is more swollen in view of the shape of the aneurysm.

Referring to the third line of FIG. 3, the left side has a much higher aspect ratio, but the moments of inertia are similar to each other, and the possibility of rupture is similar only by the shape of the aneurysm, and thus it may be more accurate to predict the moment of inertia.

Compared to the conventional method with reference to FIG. 3, using the moment of inertia according to the exemplary embodiment of the present disclosure may predict aneurysm rupture equally or more accurately compared to the conventional method.

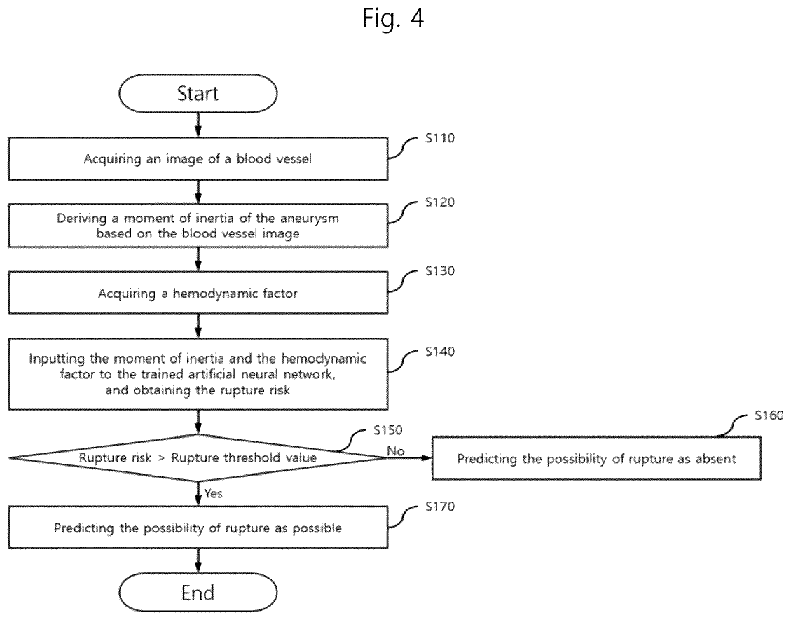

FIG. 4 is a flowchart of a method of predicting aneurysm rupture according to an embodiment of the present disclosure. A method of predicting aneurysm rupture will be described with reference to FIG. 4.

The blood vessel image acquisition processor 110 may acquire a blood vessel image (S110).

The inertia moment calculation processor 120 may derive the moment of inertia of the aneurysm based on the blood vessel image (S120).

The hemodynamic factor acquisition processor 130 may acquiring a hemodynamic factor about the blood vessel (S130).

The hemodynamic factor may include at least one of blood flow rate, systolic blood pressure, diastolic blood pressure, and vascular wall elasticity.

The rupture prediction processor 160 may input the moment of inertia and the hemodynamic factor to the trained artificial neural network 140, and may also obtain the rupture risk from the artificial neural network 140 (S140).

The artificial neural network 140 may be trained in advance to output the rupture risk based on the previously collected data of the moment of inertia, the hemodynamic factor, and the possibility of rupture.

The rupture prediction processor 160 may compare the rupture risk and the rupture threshold value (S150).

The rupture threshold value may be derived based on the ROC curve for the artificial neural network 140 in the threshold value setting processor 150.

When the rupture risk is not greater than the rupture threshold value, the rupture prediction processor 160 may predict the possibility of rupture as absent (S160).

When the rupture risk is greater than the rupture threshold value, the rupture prediction processor 160 may predict the possibility of rupture as possible (S170).

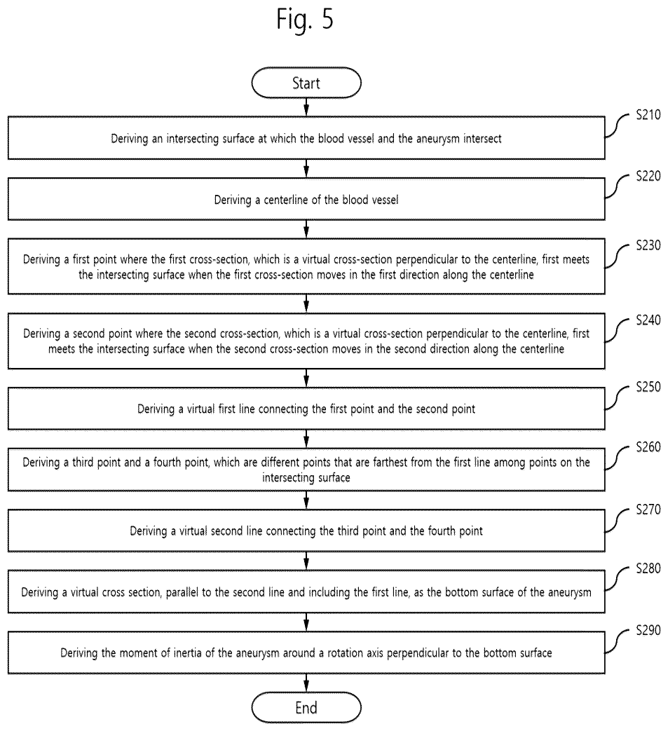

FIG. 5 is a flowchart of a method of deriving a moment of inertia of an aneurysm, in accordance with an embodiment of the present disclosure. A method of deriving the moment of inertia of the aneurysm will be described in detail with reference to FIG. 5.

The inertia moment calculation processor 120 may derive an intersecting surface between the blood vessel and the aneurysm (S210).

The inertia moment calculation processor 120 may derive a centerline, which is a virtual line passing through the center of the blood vessel (S220).

The inertia moment calculation processor 120 may derive a first point where the first cross-section, which is a virtual cross-section perpendicular to the centerline, first meets the intersecting surface when the first cross-section moves in the first direction along the centerline (S230).

The first direction may be a direction from one end of the blood vessel toward the aneurysm.

The inertia moment calculation processor 120 may derive a second point where the second cross-section, which is a virtual cross-section perpendicular to the centerline, first meets the intersecting surface when the second cross-section moves in the second direction along the centerline (S240).

The second direction may be a direction different from the first direction and may be a direction from the other end of the blood vessel, which is different from the one end of the blood vessel toward the aneurysm.

The inertia moment calculation processor 120 may derive a virtual first line connecting the first point and the second point (S250).

The inertia moment calculation processor 120 may derive third point and a fourth point, which are different points that are farthest from the first line among points on the intersecting surface (S260).

The third point may be a point located at an edge of any one surface of the intersecting surface divided by the first line.

The fourth point may be a point located at an edge of a surface different from a surface where the third point is located among the intersecting surfaces divided by the first line.

The inertia moment calculation processor 120 may derive a virtual second line connecting the third point and the fourth point (S270).

The inertia moment calculation processor 120 may derive a virtual cross section, parallel to the second line and including the first line, as the bottom surface of the aneurysm (S280).

The inertia moment calculation processor 120 may derive the moment of inertia of the aneurysm around a rotation axis perpendicular to the bottom surface (S290).

FIG. 6 is a configuration diagram of an artificial neural network according to an embodiment of the present disclosure. An artificial neural network will be described with reference to FIG. 6.

The artificial neural network may include an input layer, a hidden layer, and an output layer. The artificial neural network may include one or more hidden layers. The more the hidden layer is included, the more accurate training is possible, so accurate output can be expected, but since the time required for calculation increases, the infinite hidden layer cannot be increased.

The artificial neural network can be trained so that a desired output comes out for an input. Such training may be performed by adjusting a weight of an active function in the hidden layer through backpropagation.

A framework implementing an artificial neural network is widely used in the technical field to which the present disclosure belongs. For example, only the artificial neural network may be implemented using TensorFlow, Keras, Theano, Pytorch, Apache MXNet, My Microsoft CNTK, or the like.

According to an embodiment of the present disclosure, when receiving the moment of inertia and the hemodynamic factor, the artificial neural network 140 may output a value indicating the rupture risk.

The hemodynamic factor may be one or more values, and accordingly, the artificial neural network 140 may receive two or more data.

The rupture risk may be a value in the range of 0 to 1.

In order to train the artificial neural network 140, the moment of inertia and the hemodynamic data having a rupture risk of 1 and the moment of inertia and the hemodynamic data having a rupture risk of 0 may be input to the artificial neural network 140.

FIG. 7 is an illustration of a receiver operating characteristic (ROC) curve, according to an embodiment of the present disclosure. The use of the ROC curve to set the rupture threshold value will be described with reference to FIG. 7.

The ROC curve is mainly used in medicine and is used as a method of evaluating the usefulness of an inspection method. With the emergence of various models for artificial intelligence, it is also used as a means to evaluate the accuracy of artificial intelligence models.

As illustrated in FIG. 7, the ROC curve may be a graph in which a y-axis has a value of sensitivity and an x-axis has a value of 1-specificity. As an example of sensitivity and specificity, the sensitivity may indicate an actual ruptured rate of aneurysm predicted to be ruptured, and the specificity may indicate an actual unruptured rate of aneurysm predicted not to be ruptured. According to this, in the case of a 100% accurate artificial neural network, both sensitivity and specificity may be 1.

The area of the lower region of the graph in the ROC curve is referred to as an area under the curve (AUC), and the larger the AUC, the more it can be evaluated as a perfect inspection method. For example, when both the sensitivity and the specificity are 1, the AUC has a value of 1.

When the artificial neural network is trained based on the previously collected moment of inertia, the hemodynamic factor, and the rupture risk, the ROC curve related to the artificial neural network may be created.

As the training progresses, the artificial neural network outputs a more accurate rupture risk value, and accordingly, the AUC may approach 1.

Referring to FIG. 7, an ROC curve indicated by AUC 0.88 may indicate an artificial neural network that is more accurate than AUC 0.75, and may indicate an artificial neural network that is more trained.

The threshold value setting processor 150 may create a ROC curve of the trained artificial neural network 140 and derive a rupture threshold value based on the ROC curve.

According to an embodiment of the present disclosure, the threshold value setting processor 150 may derive the sensitivity of the point of the ROC curve closest to the coordinates (0, 1) of the ROC curve graph as the rupture threshold value.

According to another embodiment of the present disclosure, the threshold value setting processor 150 may derive the largest value among the sensitivities at the point where the ROC curve and the straight line having the slope of 1 intersect as the rupture threshold value.

It will be understood by those skilled in the art related to the embodiments of the present disclosure that the present disclosure may be implemented in a modified form without departing from the essential characteristics of the disclosure. Therefore, the disclosed methods should be considered from a descriptive point of view, not a restrictive point of view. The scope of the present disclosure appears in Claims, not in the detailed description of the invention, and all differences within the scope equivalent thereto should be interpreted as being included in the scope of the present disclosure

### INDUSTRIAL AVAILABILITY

The present disclosure relates to a method for predicting the aneurysm rupture by using artificial intelligence based on the morphological factor and the hemodynamic factor of an aneurysm, and to a device for the same, which can derive the rupture risk by inputting the morphological factor and the hemodynamic factor of an aneurysm into an artificial neural network, and can predict the possibility of rupture based on the derived rupture risk, and can more accurately predict the possibility of an atypical-shaped aneurysm rupture by deriving the moment of inertia of the aneurysm based on the morphological factor and using the derived moment of inertia, thereby having high industrial applicability.

1. A method for predicting aneurysm rupture comprising the steps of:
acquiring an image of a blood vessel;
deriving moment of inertia based on the image of the blood vessel;
acquiring a hemodynamic factor;
outputting the rupture risk when the moment of inertia and the hemodynamic factor are inputted to a pre-trained artificial neural network; and
predicting the possibility of rupture as possible when the rupture risk is greater than a predetermined rupture threshold value, and predicting the possibility of rupture as absent when the rupture risk is not greater than the predetermined rupture threshold value.

2. The method for predicting aneurysm rupture of claim 1,
wherein the deriving moment of inertia further comprises:
deriving an intersecting surface at which the blood vessel and the aneurysm intersect;
deriving a centerline of the blood vessel;
deriving a first point where the first cross-section, which is a virtual cross-section perpendicular to the centerline, first meets the intersecting surface when the first cross-section moves in the first direction along the centerline;
deriving a second point where the second cross-section, which is a virtual cross-section perpendicular to the centerline, first meets the intersecting surface when the second cross-section moves in the second direction along the centerline;
deriving a virtual first line connecting the first point and the second point;
deriving a third point and a fourth point, which are different points that are farthest from the first line among points on the intersecting surface;
deriving a virtual second line connecting the third point and the fourth point;
deriving a virtual cross section, parallel to the second line and including the first line, as the bottom surface of the aneurysm; and
deriving the moment of inertia of the aneurysm around a rotation axis perpendicular to the bottom surface.

3. The method for predicting aneurysm rupture of claim 2,
wherein the first direction is a direction from one end of the blood vessel toward the aneurysm,
the second direction is a direction from the other end of the blood vessel, which is different from the one end of the blood vessel toward the aneurysm,
the third point is a point located at an edge of one surface of the intersecting surface divided by the first line, and
the fourth point is a point located at an edge of another surface of the intersecting surface divided by the first line, which is different from the one surface on which the third point is located.

4. The method for predicting aneurysm rupture of claim 1,
wherein the deriving moment of inertia further comprises:
normalizing the moment of inertia using factors related to the shape of the aneurysm and the blood vessel comprising a diameter of the blood vessel, a horizontal length, vertical length and a volume of the aneurysm, and a Neck (aneurysm entrance surface) area.

5. The method for predicting aneurysm rupture of claim 1,
wherein the deriving the moment of inertia further comprises:
calculating the moment of inertia using a r³ (skewness) or a r⁴ (kurtosis).

6. The method for predicting aneurysm rupture of claim 1,
wherein the hemodynamic factor includes at least one of blood flow rate, systolic blood pressure, diastolic blood pressure, and vascular wall elasticity.

7. The method for predicting aneurysm rupture of claim 1,
further comprising before the acquiring an image of a blood vessel:
training the artificial neural network based on data of previously prepared moments of inertia, hemodynamic factors, and rupture risk.

8. The method for predicting aneurysm rupture of claim 1,
further comprising before the acquiring an image of a blood vessel:
generating a receiver operating characteristic (ROC) curve verifying accuracy of a rupture risk output from the pre-trained artificial neural network; and
deriving the rupture threshold value based on the ROC curve.

9. The method for predicting aneurysm rupture of claim 8,
wherein the deriving the rupture threshold value comprises:
deriving a point of the ROC curve closest to coordinates (0, 1) of the ROC curve graph; and
deriving a sensitivity of the point of the ROC curve as the rupture threshold value.

10. The method for predicting aneurysm rupture of claim 8,
wherein the deriving of the rupture threshold value comprises:
obtaining an intersecting point at which a straight line having a slope of 1 intersects the ROC curve; and
deriving a largest value among sensitivities of the intersecting point as the rupture threshold value.

11. A device for predicting aneurysm rupture comprising:
a blood vessel image acquisition processor for acquiring an image of a blood vessel;
an inertia moment calculation processor for deriving an intersecting surface at which the blood vessel and an aneurysm intersect based on the image of a blood vessel, and deriving moment of inertia of the aneurysm based on the intersecting surface;
a hemodynamic factor acquisition processor for acquiring a hemodynamic factor including at least one of a blood flow rate, a systolic blood pressure, a diastolic blood pressure, and vascular wall elasticity;
an artificial neural network trained based on data of a previously collected moments of inertia, hemodynamic factors, and rupture risk;
a threshold value setting processor for deriving a rupture threshold value based on an ROC curve generated using the artificial neural network; and
a rupture prediction processor for inputting the derived moment of inertia and the acquired hemodynamic factor in the artificial neural network, and predicting the possibility of the aneurysm rupture as possible when the rupture risk output from the artificial neural network is greater than a predetermined rupture threshold value.

12. The device for predicting aneurysm rupture of claim 11,
wherein inertia moment calculation processor is configured to:
derive an intersecting surface at which the blood vessel and the aneurysm intersect;
derive a centerline of the blood vessel;
derive a first point where the first cross-section, which is a virtual cross-section perpendicular to the centerline, first meets the intersecting surface when the first cross-section moves in the first direction along the centerline;
derive a second point where the second cross-section, which is a virtual cross-section perpendicular to the centerline, first meets the intersecting surface when the second cross-section moves in the second direction along the centerline;
derive a virtual first line connecting the first point and the second point;
derive a third point and a fourth point, which are different points that are farthest from the first line among points on the intersecting surface;
derive a virtual second line connecting the third point and the fourth point;
derive a virtual cross section, parallel to the second line and including the first line, as the bottom surface of the aneurysm;
derive the moment of inertia of the aneurysm around a rotation axis perpendicular to the bottom surface;
wherein the first direction is a direction from one end of the blood vessel toward the aneurysm,
the second direction is a direction from the other end of the blood vessel, which is different from the one end of the blood vessel toward the aneurysm,
the third point is a point located at an edge of one surface of the intersecting surface divided by the first line, and
the fourth point is a point located at an edge of another surface of the intersecting surface divided by the first line, which is different from the one surface on which the third point is located.

13. The device for predicting aneurysm rupture of claim 11,
wherein the inertia moment calculation processor normalizes the moment of inertia using factors related to the shape of the aneurysm and the blood vessel comprising a diameter of the blood vessel, a horizontal length, vertical length and a volume of the aneurysm, and a Neck (aneurysm entrance surface) area.

14. The device for predicting aneurysm rupture of claim 11,
wherein inertia moment calculation processor calculates the moment of inertia using a r³ (skewness) or a r⁴ (kurtosis).
